SEQUENCE LISTING (7 sequences)

SEQ ID NO: 1 - protein, 17 aa, Artificial sequence
  Synthetic peptide
YGSLPQKSQRSQDENPV

SEQ ID NO: 2 - DNA, 22 bp, Artificial sequence
  Single strand DNA oligonucleotide
atggctgccatgtcagaagaag

SEQ ID NO: 3 - DNA, 25 bp, Artificial sequence
  Single strand DNA oligonucleotide
ctaactttgatgtaagttagtaaga

SEQ ID NO: 4 - DNA, 24 bp, Artificial sequence
  Single strand DNA oligonucleotide
tactgccaaggcacactcattgaa

SEQ ID NO: 5 - DNA, 23 bp, Artificial sequence
  Single strand DNA oligonucleotide
cgcttccttaggctagattctgg

SEQ ID NO: 6 - DNA, 21 bp, Artificial sequence
  Single strand DNA oligonucleotide
catcgtgggccgctctaggca

SEQ ID NO: 7 - DNA, 21 bp, Artificial sequence
  Single strand DNA oligonucleotide
ccggccagccaagtccagacg

(19) United States
(12) Patent Application Publication
(10) Pub. No.: US 2004/0213786 A1
(43) Pub. Date: Oct. 28, 2004

(54) Compositions and methods for treating T-cell mediated autoimmune diseases

(75) Inventors: Nathan Karin, Haifa (IL); Gizi Wildbaum, Klryat Yam (IL)

Correspondence Address: G.E. EHRLICH (1995) LTD., c/o ANTHONY CASTORINA, 2001 JEFFERSON DAVIS HIGHWAY, SUITE 207, ARLINGTON, VA, 22202, US

(73) Assignee: Rappaport Family Institute for Research in the Medical Sciences

(21) Appl. No.: 10/749,515

(22) Filed: Jan. 2, 2004

Related U.S. Application Data
(63) Continuation-in-part of application No. 09/856,798, filed on Aug. 24, 2001, now abandoned.
Application No. 09/856,798 is a 371 of international application No. PCT/US99/26094, filed on Nov. 4, 1999.

(51) Int. Cl. A61K 39/395; C07K 16/24

(52) U.S. Cl. 424/145.1; 530/388.23

(57) ABSTRACT

Methods for treating autoimmune diseases are provided. The methods utilize antibodies, antibody fragments or antigenic portions of IGIF or polynucleotides encoding each for inducing protective immunity against the T-cell mediated diseases in individuals having or being predisposed to such diseases.

This application is a continuation-in-part of U.S. patent application Ser. No. 09/856,798, filed 24 Aug 2001, which is a national phase of PCT/US99/26094, Filed 4 Nov 1999, which claims priority from U.S. patent application Ser. No. 09/200,716, file 27 Nov 1998, now abandoned.

### FIELD AND BACKGROUND OF THE INVENTION

The present invention relates to the treatment of T-cell mediated autoimmune diseases, such as multiple sclerosis and arthritis and, more particularly, to interferon gamma inducing factor (IGIF) based vaccines for effecting same.

Based on their cytokine profile CD4⁺T-cells can be divided to Th1 cells that produce large amounts of interferon gamma (IFN-γ) and TNF-α, and, to a much lesser extent, IL4 and IL-10; Th2 cells that produce IL4, IL-10, and IL-13 and, to a much lesser extent, IFN-γ and TNF-α (1-10), and the newly defined Th3 cells that produce significant amounts of transforming growth factor beta (TGF-β) and have been associated with oral tolerance (11).

Th1 cells selected in response to various auto-antigens transfer T-cell mediated autoimmune diseases, whereas IL4 secreting Th2 cells, selected in response to these same antigens, either inhibit or exert no profound effect on the inflammatory process (5, 12-24).

High levels of IFN-γ and low levels of IL-4 positively select for Th1 cells, whereas, low levels of IFN-γ together with high levels of IL-4 mediate Th2 selection (1-6).

Interferon gamma inducing factor (IGIF, interleukin-18, IL-18) is a recently described cytokine (25) that shares structural features with the interleukin-l (IL-1) family of proteins (26). Activation of IGIF is mediated by interleukin-1 beta converting enzyme (ICE) (27, 28). Like IL-12, IGIF is a potent inducer of the production of IFN-γby Th1 and natural killer (NK) cells, and acts on Th1 cells together with IL-12 in a synergistic manner (25, 29-32).

Experimental autoimmune encephalomnyelitis (EAE) is a T-cell mediated autoimmune disease of the central nervous system (CNS) which, for many years and for a variety of experimental protocols, serves as a model for the human disease, multiple sclerosis (MS), a chronic degenerative disease marked by patchy destruction of the myelin that surrounds and insulates nerve fibers and mild to severe neural and muscular impairments, since in both diseases circulating leukocytes penetrate the blood brain barrier and damage myelin resulting in impaired nerve conduction and paralysis (33, 34). Antigen specific T-cells are thought to play a pivotal role in the manifestation of both diseases (35-37).

The role of Th1 cells in the manifestation of EAE has been widely studied. Th1 but not Th2 cells transfer the disease to normal naive recipients (18). Shifting the Th1/Th2 balance towards Th2 cells by in vivo administration of IL-4 (12), by antibodies to B7-1 (14), by soluble peptide therapy (38), or by administration of neutralizing antibodies to IL-12 (15) markedly suppressed EAE.

It has recently been shown that IGIF is a more potent inducer of IN-γ producing Th1 cells than is IL-12 and thus plays an important role in Th1 responses (25). However, the possible role of anti-IGIF immunotherapy in regulation of T-cell mediated autoimmunity has never been evaluated.

While reducing the present invention to practice it has been shown, for the first time, that neutralizing antibodies to IGIF ameliorate EAE and AA, thus indicating that IGIF neutralization can be utilized. to treat T-cell mediated autoimmune diseases.

### SUMMARY OF THE INVENTION

The present invention discloses the use of anti interferon gamma inducing factor antibody in the treatment of T-cell mediated autoimmune diseases such as multiple sclerosis. This use can be effected in a variety of ways as further described and exemplified hereinbelow.

According to one aspect of the present invention there is provided an antibody or antibody fragment capable of specifically neutralizing an interferon gamma inducing factor.

According to another aspect of the present invention there is provided a method of inducing protective immunity against a T-cell mediated autoimmune disease, the method comprising administering to an individual having the T-cell mediated autoimmune disease or being predisposed thereto, cells being capable of producing and secreting an antibody capable of neutralizing an interferon gamma inducing factor thereby inducing protective immunity against the Tell mediated autoimmune disease in the individual.

According to yet another aspect of the present invention there is provided an article-of-manufacturing comprising packaging material and a pharmaceutical composition being identified for use in inducing protective immunity against a T-cell mediated autoimmune disease, the pharmaceutical composition comprising a pharmaccutically acceptable carrier and an antibody being capable of binding an interferon gamma inducing factor.

According to a farther aspect of the present invention there is provided method of inducing protective immunity against a T-cell mediated autoimmune disease, the method comprising administering to an individual having the T-cell mediated autoimmune disease or being predisposed thereto an antibody capable of neutralizing an interferon gamma inducing factor thereby inducing protective immunity against the T-cell mediated autoimmune disease in the individual.

According to further features in preferred embodiments of the invention described below, the antibody is monoclonal.

According to still farther features in the described preferred embodiments the antibody is humanized.

According to still further features in the described preferred embodiments the interferon gamma inducing factor is IL-18.

According to still further features in the described preferred embodiments the T-cell mediated autoimmune disease is selected from the group consisting of multiple sclerosis, rheumatoid arthritis, type I diabetes, uveoretinitis, Crohn's disease and ulcerative colitis.

According to still further features in the described preferred embodiments the T-cell mediated autoimmune disease is selected from the group consisting of multiple sclerosis. rheumatoid arthritis, type I diabetes, uveoretinitis, Crohn's disease and ulcerative colitis.

According to still another aspect of the present invention there is provided a method of inducing protective immunity against multiple sclerosis, the method comprising administering to an individual having the T-cell mediated autoimmune disease or being predisposed thereto a nucleic acid construct including a polynucleotide sequence encoding a polypeptide being capable of eliciting in the individual formation of antibodies capable of neutralizing an interferon gamma inducing factor thereby inducing protective immunity against multiple sclerosis in the individual.

According to an additional aspect of the present invention there is provided a method of inducing protective immunity against a T-cell mediated autoimmune disease, the method comprising:(a) obtaining cells of an individual; (b) introducing into the cells a nucleic acid construct including a polynucleotide sequence encoding an interferon gamma inducing factor or an immunogenic portion thereof to thereby generate genetically modified cells expressing and optionally secreting the interferon gamma inducing factor or the immunogenic portion thereof; and (c) reintroducing the genetically modified cells to the individual thereby inducing protective immunity against the T-cell mediated autoimmune disease in the individual.

According to yet an additional aspect of the present invention there is provided an article-of-manufacturing comprising packaging material and a pharmaceutical composition being identified for use in inducing protective immunity against a T-cell mediated autoimmune disease, the pharmaceutical composition comprising a pharmaceutically acceptable carrier and a nucleic acid construct including a polynucleotide sequence encoding a polypeptide being capable of eliciting formation of antibodies capable of neutralizing an interferon gamma inducing factor.

According to still fiercer features in the described preferred embodiments the nucleic acid construct also includes one or more transcription control sequences operatively linked to the polynucleotide sequence.

According to still further features in the described preferred embodiments the transcription control sequences are selected from the group consisting of RSV control sequences, CMV control sequences, retroviral LTR sequences, SV-40 control sequences and β-actin control sequences.

According to still further features in the described preferred embodiments the nucleic acid construct is an eukaryotic expression vector.

According to still further features in the described preferred embodiments the nucleic acid construct is selected from the group consisting of pcDNA3, pcDNA3.1(+/−), pZeoSV2(+/−), pSecTag2, pDisplay, pEF/myc/cyto, pCMV/inyclcyto, pCR3.1, pCI, pBK-RSV, pBK-CMV, pTRES and their derivatives.

According to still further features in the described preferred embodiments the nucleic acid construct is administered to the individual parenterally.

According to still further features in the described preferred embodiments the individual is a human being.

According to still further features in the described preferred embodiments the nucleic acid construct is administered to the individual via a viral vector.

According to still further features in the described preferred embodiments the delivery vehicle is selected from the group consisting of liposomes, micelles, and cells.

According to still further features in the described preferred embodiments the nucleic acid construct is an eukaryotic expression vector.

According to still further features in the described preferred embodiments the pharmaceutical composition is formulated suitable for parenteral administration to a human.

According to still an additional aspect of the present invention there is provided use of an anti interferon gamma inducing factor antibody in the treatment of a T-cell mediated autoimmune disease.

According to still further features in the described preferred embodiments the T-cell mediated autoimmune disease is selected from the group consisting of multiple sclerosis, rheumatoid arthritis, type I diabetes, uveoretinitis, Crohn's disease and ulcerative colitis.

The present invention successfully addresses the shortcomings of the presently known configurations by providing new and promising approaches for treatment of devastating autoimmune diseases such as multiple sclerosis.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE DRAWINGS

The invention herein described, by way of example only, with reference to the accompanying drawings, wherein:

FIGS. 1A-F demonstrate IGIF mRNA in the inflamed EAE brain. FIGS. 1A, C, G and E—Rats were injected with 10⁷ cells form L68-86 immunized rats to allow for the development of transferred EAE. Before adoptive transfer of disease (day 0), and at various time points: before the onset of disease (day 3), at the day of onset (day 5), the peak (day 7), following recovery (day 10), and 10 days after recovery (day 20) mid-brain and brain stem samples from six different rats at each time point were examined. mRNA was isolated from each sample and subjected to RT-PCR analysis using specific oligonucleotide printers constructed for IGIF (FIG. 1C) and for IFN-γ (FIG. 1E). Each amplification was calibrated to β-actin (FIG. 1G) and verified by Southern Blot analysis. Southern bolt images were objectively assessed using an FujiFilm Thermal System FIGS. 1B, D, F and H—Rats were immunized with p68-86/CFA and developed active EAE. Before the induction of disease (day 0), and at various time points: before the onset of disease (day 8), at the peak (day 13), and following recovery (day 21) mid-brain and brain stem samples from six different rats at each time point were examined for mRNA transcription for IGIF (FIG. 1D) and IFN-γ (FIG. 1F) and calibrated to β-actin (FIG. 1H), as described above.

FIGS. 2A-D demonstrate that neutralizing antibodies to recombinant rat IGIF block IFN-γ production in cultured T-cells. Spleen cells from naive (FIGS. 2A and 2B) or from p68-86/CFA primed (day 9) Lewis rats (FIGS. 2C and 2D) were cultured in vitro with either Con A (FIGS. 2A and 2B), or with 100 μM of MBP p68-86 (FIGS. 2C and 2D) with or without the addition of 100 ng/ml of rabbit anti-rat IGIF (IgG) neutralizing antibodies (FIGS. 2A and 2C) or with IgG from non-immunized rabbits (data not shown), with or without the addition of 400 ng/ml recombinant rat IGIF (FIGS. 2B and 2D). After 72 hours of stimulation IFN-γ levels were determined in the culture supernatants by an ELISA assay. Results are the mean ±S.E. of triplicate cultures.

FIGS. 3A-B demonstrate that neutralizing antibodies to recombinant rat IGIF block the development of both active and transferred EAE. FIG. 3A—Lewis rats were immunized with p68-86/CFA to induce active EAE and then separated into three groups of six rats each. Eight, ten and eleven days after induction of disease, these groups were injected IV with rabbit anti-rat IGIF (IgG fraction 100 μg/(rat), with IgG fraction purified from non-immunized rabbits (control IgG), or with PBS. The rats were then monitored daily for clinical signs of EAE by an observer blind to the treatment protocol. Results arc presented as mean clinical score ±S.E. FIG. 3B—Transferred EAE was induced as described above (FIG. 1). Recipients were then separated into three groups of six rats each. Three, five and seven days after induction of disease these groups were injected as described above (FIG. 3A) and monitored daily for clinical signs of EAE by an observer blind to the treatment protocol. Results are presented as mean clinical score ±S.E.

FIGS. 4A-D demonstrate alteration in IFN-γ and IL-4 production in EAE rats injected with anti-IGIF neutralizing antibodies. Lewis rats were immunized with p68-86/CFA to induce active EAE and separated into three groups. Five and seven days after disease induction these groups were injected IV with either rabbit anti-rat IGIF (IgG fraction 100 μg/rat), with purified IgG from non-immunized rabbits, or with PBS. Before the onset of disease (day 9) splenic T-cells from three rats in each group were cultured with 100 μM MBP p68-86 for 72 hours in stimulation medium that was not (FIGS. 4A and 4B) or was (FIGS. 4C and 4D) supplemented with recombinant rat IL-4 (5 ng/ml). After 72 hours of simulation, IFN-γ levels were determined in culture supernatants by an ELISA assay. Results are of triplicate cultures expressed as mean ±S.E.

FIG. 8 demonstrates alteration in TNF-α production in EAE rats injected with anti-IGIF neutralizing antibodies—Levels of TNF-α were determined in supernatants obtained in experiment described in FIGS. 4A and 4B, by an ELISA assay. Results are of triplicate cultures expressed as mean ±S.E.

FIGS. 6A-B are graphs illustrating the effect of the anti IL-18 Mab of the present invention on EAE (FIG. 6a) and AA (FIG. 6b) as compared with isotype matched control mouse IgG1.

### DESCRIPTION OF THE PREFERRED EMBODIMENTS

The present invention is of interferon gamma inducing factor (IGIF) based vaccines which can be used in the treatment of T-cell mediated autoimmune diseases such as multiple sclerosis. Specifically, the present invention can be used to confer protective immunity against multiple sclerosis as well as arthritis and other autoimmune diseases.

The principles and operation of the interferon gamma inducing factor (IGIF) based vaccines according to the present invention may be better understood with reference to the drawings and accompanying descriptions.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

In autoimmune conditions, T-cells reactive to self-antigens escape elimination in the thymus, and are activated in the periphery where they can provoke damage to specific cells and organs. Perturbation of the balance between self-reactive T-cells with different cytokine profiles may serve as an effective way of restraining the harmful defect of autoimmune T-cells (12, 14-16, 20-22, 38, 41-43).

Cytokines present at the initiation of CD4⁺T-cell responses determine whether a Th1 or a Th2 response will predominate (1-6). Thus, administration of IL-4 or of antibodies to IL-12 preferentially favors Th2 selection in vivo and thus serves as a powerful way to inhibit two different T-cell mediated autoimmune diseases: EAE and IDDM (12, 15, 22)

It is well known that high levels of IFN-γ positively select for TNF-α secreting Th1 cells (6). A previous study showed that administration of anti-IL-12 neutralizing antibodies blocks EAE while inducing a marked reduction of both IFN-γ and TNF-α production (15). IFN-γ and TNF-α together then exhibit a synergistic effect on enhancing expression of adhesion molecules on endothelial cells (44), and on eliciting the inflammatory process, which can be reversed by either anti-adhesion molecule immunotherapy (45, 46), or by blocking TNF-α (44, 47-50). It was previously demonstrated that EAE resistance acquired by soluble antigen therapy can be reversed by anti-IL-4 neutralizing antibodies (38). This further demonstrated the pivotal role of the Th1/Th2 balance in regulation of T-cell mediated autoimmunity (38).

IGIF is a recently described cytokine (25) that shares structural features with the interleukin-1 (IL-1) family of proteins (26). Activation of IGIF is mediated by interleukin-1 beta converting enzyme (ICE) (27, 28). Like IL-12, IGIF is a potent inducer of IFN-γ from Th1 and NK cells, and acts on Th1 cells together with IL-12 in a synergistic manner (25, 29-32). IGIF actually has more potent IFN-γ inducing capabilities than IL-12 and apparently utilizes a distinct signal transduction pathway for its elicitation (25, 31, 32, 51). Little is known about the role of TGIF in T-cell mediated autoimmune disease. A recent study used RT PCR to demonstrate that the active stage of autoimmune diabetes in NOD mice is associated with the expression of IGIF (52).

As further detailed in the Examples section below, while conceiving and reducing the present invention to practice, specific oligonucleotide primers were used to identify and isolate interferon gamma inducing factor (IGIF) from the brain of rats with developing experimental autoimmune encephalomyelitis (EAE), a T-cell mediated autoimmune disease of the central nervous system (CNS) that serves as a model for multiple sclerosis (MS).

IGIF was highly transcribed in the brain at the onset and during the course of active EAE. PCR products encoding rat IGIF were used to generate the recombinant protein which was used to induce anti-IGIF neutralizing antibodies. These antibodies significantly reduced the production of interferon gamma (IFN-γ) by primed T-cells proliferating in response to their target myelin basic protein (MBP) epitope and by Con A activated T-cells from naive donors.

When administered to rats during the development of either active or transferred EAE, these antibodies significantly blocked the development of disease.

Splenic T-cells form protected rats were cultured with the encephalitogenic MBP epitope and evaluated for production of IL-4 and IFN-γ. These cells, which proliferated, exhibited a profound increase in IL-4 production, accompanied by a significant decrease in IFN-γ and TNF-α production.

An elevated expression of IGIF at the time when the secondary influx of autoimmune cells is apparent at the site of inflammation in the EAE brain (38, 39, 46, 53) is demonstrated herein for the first time. So are neutralizing antibodies, which were generated against IGIF cloned from this site of inflammation, to block the disease by altering the in vivo Th1/Th2 balance in flavor of Th2 selection. This alteration included a marked reduction in the production of IFN-γ, and, most importantly, TNF-α, a proinflammatory cytokine that plays a critical role in T-cell mediated autoimmunity (44, 47-50).

An interesting observation is that both the inhibitory effect of IGIF neutralizing antibodies and the augmentation by IGIF of IFN-γ production are more profound on activated T-cells from a naive donor than on primed T-cells responding to their target epitope.

The direct role of IFN-γ in EAE is enigmatic. Grewal et al. have used a CD40L-deficient mice that carry a transgenic T-cell receptor specific for MBP to demonstrate that EAE induction is IFN-γ dependent (54). On the other hand not only were mice lacking IFN-γ susceptible to induction of active EAE (55) but also antibodies to IFN-γ were found capable of enhancing his disease (56, 57). A recent study has demonstrated that IL-12 is directly involved in the generation of autoreactive Th1-cells that induce EAE, both in the presence and the absence of IFN-γ (58). However, it could well be that alteration the Th1/Th2 balance towards IL4 secreting Th2 cells confers EAE resistance not because it leads to a reduced production of IFN-γ, but rather because it results in a reduced production of TNF-α accompanied by a marked increase in IL-4 production.

It has recently been suggested that IGIF primarily effects IFN-γ production by Th1 but not Th2 cells (29). It is possible that immunization with p68-86/CFA induces a substantial selection of antigen specific Th2 cells, albeit not enough to inhibit the subsequent development of a Th1 mediated autoimmune disease.

As is shown in Example 1 of the Examples section which follows, in vivo neutralization of IGIF notably shifts the Th1/Th2 balance in antigen specific proliferating T-cells towards Th2, thus preventing the formation and/or progression of a T-cell mediated autoimmune response. As is shown in Example 2 of the Examples section, such a shift in the Th1/Th2 balance, substantially reduces the severity of autoimmune diseases such as multiple sclerosis and arthritis thus indicating that in-vivo IGIF neutralization can be utilized to treat T-cell mediated autoimmune diseases.

Thus, the present invention teaches the use of an anti interferon gamma inducing factor in treatment of individuals having, or being predisposed to, T-cell mediated autoimmune diseases such as multiple sclerosis, rheumatoid arthritis, type I diabetes, uveoretinitis, Crohn's disease and ulcerative colitis.

Individuals having a T-cell mediated autoimmune disease are those which have been diagnosed with the disease or exhibit symptoms related to the disease, while individuals predisposed to a T-cell mediated autoimmune disease are those susceptible to developing the disease due to age, genetic background, exposure to specific environmental conditions, or appearance of early signs that might suggest that onset of the disease is likely.

Treatment of T-cell mediated autoimmune diseases according to the present invention encompasses reducing the severity or duration of the disease and/or reducing symptoms associated with the diseases. Such treatment can be effected using various approaches, some of which are further described and exemplified hereinbelow.

According to one aspect of the present invention there is provided an antibody which comprises an immunoglobulin capable of binding and preferably neutralizing interferon gamma inducing factor (IGIF, IL-18).

As used herein in the specification and in the claims section below, the terms “antibody” and “immunoglobulin”, which are interchangeably used, refer to any of several classes of structurally related proteins that function as part of the immune response of an individual, which proteins include IgG, IgD, IgE, IgA, IgM and related proteins. These terms further relate to chimeric immunoglobulins which are the expression products of fused genes derived from different species. These terms further relate to immunologically active derivatives of the above proteins, including, but not limited to, an F(ab′)₂ fragment, an Fab fragment, an Fv fragment, a heavy chain, a light chain, an unassociated mixture of a heavy chain and a light chain, a heterodimer consisting of a heavy chain and a light chain, a catalytic domain of a heavy chain, a catalytic domain of a light chain, a variable fragment of a light chain, a variable fragment of a heavy chain, and a single chain variant of the antibody. Under normal physiological conditions antibodies are found in plasma and other body fluids and in the membrane of certain cells and are produced by lymphocytes of the type denoted B cells or their functional equivalent. Antibodies of the IgG class are made up of four polypeptide chains linked together by disulfide bonds. The four chains of intact IgG molecules are two identical heavy chains referred to as H-chains and two identical light chains referred to as L-chains. The immunoglobulin or antibody according to the present invention could also be a “humanized” antibody, in which, for example individual (say murine) variable regions ate fused to human constant regions, or in which murine complementary-determining regions are grafted onto a human antibody structure (Wilder, R. B. et al., J. Clin. Oncol., 14:1383-1400, 1996). Unlike, for example, individual derived antibodies, ‘humanized’ antibodies often do not undergo an undesirable reaction with the immune system of the subject. The terms “sFv” and “single chain antigen binding protein” refer to a type of a fragment of an immunoglobulin, an example of which is sFv CC49 (Larson, S. M. et al., Cancer, 80:2458-68, 1997). As used herein, the term “humanized antibodies” also reads on antibodies produced by non-human cells or organisms genetically modified to include nucleic acid sequences encoding a functional portion of the human immune system, wherein the resulting antibodies are substantially identical to human antibodies in that they are encoded by human derived genes. However, the term “antibody”, as used herein, further relates to soluble portions of receptors capable of specifically binding their respective protein ligands, which, in that respect, function like immunoglobulins.

As used herein and in the claims, the term “individual” refers to any organism with an immune system.

According to yet another aspect of the present invention there is provided a pharmaceutical composition for inducing protective immunity against multiple sclerosis. The composition comprises a pharmaceutically acceptable carrier and an antibody being capable of binding an interferon gamma inducing factor.

Alternatively, the composition according to the present invention comprises a pharmaceutically acceptable carrier and an interferon gamma inducing factor or an immunogenic portion thereof, thereby eliciting an antibody being capable of binding the interferon gamma inducing factor in vivo.

As used herein the phrase “immunogenic portion” refers to an immunogenic pertinacious compound, which may include, among optional additional components, a plurality of amino acid residues. The term “amino acid” is understood to include the 20 naturally occurring amino acid residues; those amino acid residues often modified post-translationally in vivo, including for example hydroxyproline, phosphoscrine and phosphotbreonine; and other unusual amino acid residues including, but not limited to, 2-aminoadipic acid, hydroxylysine, isodesmosine, nor-valine, nor-leucine and ornithine. Furthermore, the term “amino acid” includes both D- and L-amino acid residues. The amino acid residues according to the present invention form a peptide. The latter is understood to include native peptides, including degradation products or synthetically synthesized peptides, and further to peptidomimetics, such as peptoids and semipeptoids, which are peptide analogs, which may have, for example, modifications rendering the peptides more stable or less immunogenic while contacting body fluids. Such modifications include, but are not limited to, cyclization, N terminus modification, C terminus modification, peptide bond modification, including, but not limited to, CH₂—NH, CH₂—S, CH₂—S═O, O═C—NH, CH2—O, CH₂—CH₂, S═C—NH, CH═CH or CF═CH, backbone modification and residue modification. Methods for preparing peptidomimetic compounds are well known in the art and are specified in Quantitative Drug Design, C. A. Ramsden Gd., Chapter 17.2, F. Choplin Pergamon Press (1992), which is incorporated by reference as if fully set forth herein.

According to yet additional aspect of the present invention there is provided a method of inducing protective immunity against a T-cell mediated autoimmune disease. The method is effected by administering, to an individual having the disease or being predisposed thereto, an antibody capable of in vivo neutralizing an interferon gamma inducing factor.

According to a preferred embodiment of the present invention, the antibody is polyclonal. Preparation of polyclonal antibodies is known in the art and further described in the Examples section hereinunder.

According to another preferred embodiment of the present invention, the antibody is monoclonal. Methods of producing and identifying monoclonal antibodies are well known in the art.

Monoclonal antibodies may be obtained by processes comprising the generation of a plurality of monoclonal antibodies to an antigen and screening the plurality of antibodies so generated to identify a monoclonal antibody that binds and/or neutralizes the peptide of interest, interferon gamma inducing factor in the present case. Monoclonal antibodies may be generated either in vitro or in vivo. In a related process, an individual is immunized with an antigen thereby generating antibody producing lymphocytes in said individual, antibody producing lymphocytes are removed from the individual, said lymphocytes arc fused with myeloma cells to produce a plurality of immortalized hybridoma cells each of which produces monoclonal antibodies, the plurality of monoclonal antibodies is screened to identify a monoclonal antibody that binds the peptide, and the bybridoma producing the antibody is cloned and propagated. Individuals are typically immunized with a mixture comprising a solution of the immunogen in a physiologically acceptable vehicle, and any suitable adjuvant, which achieves an enhanced immune response to the immunogen. By way of example, the primary immunization conveniently may be accomplished with a mixture of a solution of the immunogen and Freud's complete adjuvant, said mixture being prepared in the form of a water in oil emulsion. Typically the immunization may be administered to the individuals intram uscularly, intradermally, subcutaneously, intrapertoneally, into the footpads, or by any appropriate route of administration. The immunization schedule of the immunogen may be adapted as required, but customarily involves several subsequent or secondary immunizations using a milder adjuvant such as Freund's incomplete adjuvant. Antibody titers and specificity of binding to the hapten can be determined during the immunization schedule by any convenient method including by way of example radioimmunoassay, or enzyme linked immunoassay. Antibody activity assays can performed in vitro as further exemplified in the Examples section that follows. When suitable antibody titers are achieved, antibody producing lymphocytes from the immunized individuals are obtained, and these are cultured, selected and cloned, as is known in the art. Typically, lymphocytes may be obtained in large numbers from the spleens of immunized individuals, but they may also be retrieved from the circulation, the lymph nodes or other lymphoid organs. Lymphocytes are then fused with any suitable myeloma cell line, to yield hybridomas, as is well known in the art. Alternatively, lymphocytes may also be stimulated to grow in culture, and may be immortalized by methods known in the art including the exposure of these lymphocytes to a virus, a chemical or a nucleic acid such as an oncogene, according to established protocols. After fusion, the hybridomas are cultured under suitable culture conditions, for example in multiwell plates, and the culture supernatants are screened to identify cultures containing antibodies that recognize the hapten of choice. Hybridomas that secrete antibodies that recognize the hapten of choice are cloned by limiting dilution and expanded, under appropriate culture conditions. Monoclonal antibodies are purified and characterized in terms of immunoglobulin type binding affinity and in vivo or in vitro neutralizing activity.

Example 2 of the Examples section which follows detail preparation and characterization of an anti IL-18 monoclonal antibody.

The antibody according to the present invention, be it a poly- or monoclonal antibody, is a neutralizing antibody to interferon gamma inducing factor in affecting cells to produce interferon gamma, that is to say that the antibody interferes with the functionality of interferon gamma inducing factor in affecting cells to produce interferon gamma.

Since induction of protective immunity can also be effected by eliciting the body of the individual to produce anti-IGIF antibodies, according to still additional aspect of the present invention inducing protective immunity against a T-cell mediated autoimmune disease can also be effected by administering, to the individual, an antigen including an interferon gamma inducing factor or an immunogenic portion thereof.

As used herein the term “antigen” refers to an immunogen including at least one immunogenic epitope, which is represented in the equivalent native peptide in a continuous or discontinuous fashion. Antigenic portions of the IGIF IL-18 can be determined using software applications well known to the ordinary skilled artisan.

Yet another approach for inducing protective immunity utilizes cells capable of producing and secreting an antibody capable of in vivo neutralizing an interferon gamma inducing factor. To this end, cloned cDNAs encoding antibodies or fragments thereof are used to genetically modify receptive cells (e.g., hematopoietic cells) which are harvested from the individual or obtained from a cell line (e.g., stem cell line), to thereby render the cells antibody producing cells.

Cloning of cDNAs encoding antibodies or fragments thereof may be accomplished by several approaches known in the art. In the preferred approach, mRNA from clonal hybridoma cell lines which produce antibodies is employed as starting material. The cells are harvested and mRNA is extracted by standard methods known in the art. The cDNA is prepared by reverse transcription of the mRNA by standard methods known in the art. The cDNA for each chain of the immunoglobulin is cloned separately, and may be amplified by polymerase chain reaction using appropriate primers. The cDNA is then ligated into appropriate vectors by standard methods. The cDNA may be cloned into expression vectors and expressed separately in any convenient expression system, so that the properties of the expressed single chains of the antibodies may be determined. Alternatively, the individual chains may be expressed in the same cells which are then screened for the production of recombinant active antibodies. The method of using the invention will be modified in accordance with the system that is selected according to the current principles that are known in the art of recombinant protein production. According to the present invention the genetic information for the production of the antibody of interest is introduced into the cells by an appropriate vector as is known in the art or by any other acceptable means. The present invention provides information that will enable the skilled artisan to prepare constructs of genetic material comprising an open reading frame that encodes at least one chain of a novel antibody capable of binding IGIF. It will be appreciated that in certain embodiments it will suffice to produce an active fragment of the catalytic activity, for instance a Fab fragment of the intact antibody or even an Fv fragment thereof. In addition to the nucleic acid encoding the protein or polypeptide of choice, the constructs of the invention may comprise the following elements: a selectable marker, an origin of replication, a transcriptional promoter, a translation start site, a signal sequence for secretion of the product.

U.S. patent application Ser. No. 09/123,485, filed Jul. 28, 1998, which is incorporated by reference as if fully set forth herein, teaches the effectiveness of DNA vaccines in inducing protective immunity against multiple sclerosis. In that application, DNA sequences encoding for a variety of chemokines and for the cytokine tumor necrosis factor alpha, were shown to elicit protective immunity against both induced and transferred EAE, while the prior art teaches protective immunity against both induced and transferred EAE via passive vaccination (administration of antibodies).

Similarly, DNA vaccination in various forms, some of which are fiber detailed hereinunder, can be used to confer protective immunity against a T-cell mediated autoimmune disease.

Thus, according to still another aspect of the present invention there is provided a method of inducing protective immunity against a T-cell mediated autoimmune disease. The method is effected by administering to an individual having the disease or being predisposed thereto, a therapeutic composition which includes a nucleic acid construct including a polynucleotide sequence encoding a polypeptide capable of eliciting formation of antibodies capable of in vivo neutralizing an interferon gamma inducing factor.

Such an approach to inducing protective immunity can be effected by obtaining cells from the individual and genetically modifying the cells with a nucleic acid construct which includes a polynucleotide sequence encoding an interferon gamma inducing factor or an immunogenic portion thereof. Once such cells are tested for the ability to produce the interferon galna inducing factor or an immunogenic portion thereof, they are reintroduced into the individual.

As used herein in the specification and in the claims section below, the term “genetically modified” refers to a process of inserting nucleic acids into cells. The insertion may, for example, be effected by transformation, viral infection, injection, transfection, gene bombardment, electroporation or any other means effective in introducing nucleic acids into cells. Following the modification the nucleic acid is either integrated in all or part, to the cell's genome (DNA), or remains external to the cell's genome, thereby providing stably modified or transiently modified cells. The cells according to this method of the invention may be of any kind. Especially suitable cells are those readily removable, genetically modifiable, and reintroduceable cells, such as, but not limited to, cells of the various blood lineages, derived either from whole blood or from bone marrow, fibroblas T-cells, etc. The genetically modified cells are preferably reintroduced to the individual parenterally.

According to still another aspect of the present invention there is provided a pharmaceutical composition for inducing protective immunity against a T-cell mediated autoimmune disease. The composition comprises a pharmaceutically acceptable carrier and a nucleic acid construct including an isolated nucleic acid sequence encoding a polypeptide being capable of eliciting formation of antibodies capable of in vive neutralizing an interferon gamma inducing factor.

According to a preferred embodiment of the present invention, the therapeutic composition is administered to the individual parenterally. According to another preferred embodiment of the present invention, the individual is a human being.

According to still further features in the described preferred embodiments the pharmaceutically acceptable carrier is selected from the group consisting of an aqueous physiologically balanced solution, an artificial lipid-containing substrate, a natural lipid-containing substrate, an oil, an ester, a glycol, a virus and metal particles. Preferably, the pharmaceutically acceptable carrier comprises a delivery vehicle that delivers the nucleic acid sequences to the individual. The delivery vehicle can be effected by liposomes, micelles or cells.

According to a preferred embodiment of the present invention, the polynucleotide sequence is operatively linked to one or more transcription control sequences, such as, but not limited to, RSV control sequences, CMV control sequences, retroviral LTR sequences, SV-40 control sequences and/or β-actin control sequences. Preferably, the nucleic acid construct is an eukaryotic expression vector, such as, but not limited to, pcDNA3, pcDNA3.1(+/−), pZeoSV2(+/−), pSecTag2, pDisplay, pEF/myc/cyto, pCMV/myc/cyto, pCR3.1, pCI, pBK-RSV, pBK-CMV, pTRES, and their derivatives.

Expression vectors containing regulatory elements from eukaryotic viruses such as retroviruses can be also used. SV40 vectors include pSVT7 and pM.2. Vectors derived from bovine papillona virus include PBV-1MTHA, and vectors derived from Epstein Bar virus include pHEBO, and p2O5. Other exemplary vectors include pMSG, pAV009/A⁺, pMTO10/A⁺, pMAMneo-5, baculovirus pDSVE, and any other vector allowing expression of proteins under the direction of the SV-40 early promoter, SV-40 later promoter, metallothionein promoter, murine mammary tumor virus promoter, Rous sarcoma virus promoter, polyhedrin promoter, or other promoters shown effective for expression in eukaryotic cells.

As described above, viruses are very specialized infectious agents that have evolved, in many cases, to elude host defense mechanisms. Typically, viruses infect and propagate in specific cell types. The targeting specificity of viral vectors utilizes its natural specificity to specifically target predetermined cell types and thereby introduce a recombinant gene into the infected cell. Thus, the type of vector used by the present invention will depend on the cell type transformed. The ability to select suitable vectors according to the cell type transformed is well within the capabilities of the ordinary skilled artisan and as such no general description of selection consideration is provided herein. For example, bone marrow cells can be targeted using the human T-cell leukemia virus type I (HTLV-I).

Recombinant viral vectors are useful for in vivo expression of antibodies, antibody fragments or antigenic regions of IGIFs, since they offer advantages such as lateral infection and targeting specificity. Lateral infection is inherent in the life cycle of, for example, retrovirus and is the process by which a single infected cell produces many progeny virions that bud off and infect neighboring cells. The result is that a large area becomes rapidly infected, most of which was not initially infected by the original viral particles. This is in contrast to vertical-type of infection in which the infectious agent spreads only through daughter progeny. Viral vectors can also be produced that are unable to spread laterally. This characteristic can be useful if the deired purpose is to introduce a specified gene into only a localized number of targeted cells.

Various methods can be used to introduce the expression vector of the present invention into cells. Such methods are generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor Mich. (1995), Vectors. A Survey of Molecular Cloning Vectors and Their Uses, Butterworths, Boston Mass. (1988) and Gilboa et at. [Biotechniques 4 (6): 504-512, 1986] and include, for example, stable or transient transfection, lipofection, electroporation and infection with recombinant viral vectors. In addition, see U.S. Pat. Nos. 5,464,764 and 5,487,992 for positive-negative selection methods.

Introduction of nucleic acids by viral infection offers several advantages over other methods such as lipofection and electroporation, since higher transfection efficiency can be obtained due to the infectious nature of viruses.

Pharmaceutical compositions of the present invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as if further detailed above.

It will be appreciated that although the present invention is particularly suitable for treating T-cell mediated autoimmune diseases, it should be noted that other autoimmune disease in which abnormal T-cell activity is observed, such as, for example, Systemic lupus erythematosus (SLE), in which abnormal B-T cell activity is implicated as one disease contributing factor, can also benefit from the present therapeutic approach.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

### Example 1

### Materials and Experimental Methods

Rats: Female Lewis rats, six weeks old, were purchased from Harlan (Israel) and maintained under SPF conditions in an individual facility.

Peptide antigens: Myelin Basic Protein (MBP) p68-86, Y G S L P Q K S Q R S Q D E N P V (SEQ ID NO:1), was synthesized on a MilliGen 9050 peptide synthesizer by standard 9-fluorenylmethoxycarbonyl chemistry. Peptides were purified by high performance liquid chromatography. Structure was confirmed by amino acid analysis and mass spectroscopy. Only peptides that were greater than 95% pure were used in our study.

Immunizations and induction of active disease: Rats were immunized subcutaneously in the hind foot pads with 0.1 ml of MBP epitope 68-86 (p68-86) dissolved in PBS (1.5 mg/ml) and emulsified with an equal volume of CFA (incomplete Freund's adjuvant supplemented with 4 mg/ml heat-killed Mycobacterium tuberculosis H37Ra in oil (Difco laboratories, Inc., Detroit, Mich.). Rats were then monitored for clinical signs daily by an observer blind to the treatment protocol. EAE was scored as follows: 0, clinically normal; 1, flaccid tail; 2, hind limb paralysis; 3, front and hind limb paralysis.

Induction of transferred EAE: EAE was induced by immunizing Lewis rats (intraperitoneally) with 10⁸ activated spleen cells from EAE donors obtained as follows: Nine days after induction of active EAE, splenic cells were cultured (12×10⁶/ml) at 37° C. in humidified air containing 7.5% CO₂ for two days in stimulation medium that includes Dulbecco's modified Eagle's medium (Gibco) supplemented with 2-mercaptoethanol (5×10⁻⁵ M), L-glutamine (2 mM), sodium pyruvate (1 mM), penicillin (100 μg/ml), streptomycin (100 μg/ml), 1% syngeneic serum and 20-30 μg/ml of the immunizing epitope. Then, cells were separated on a Ficoll gradient (Sigma), resuspended in PBS and injected to naive recipients.

Antigen-specific T-cell proliferation assays: Lewis rats were immunized with MBIP p68-86/CFA as described above. Nine to ten days later spleen cells were suspended in stimulation medium and cultured in U-shape 96-well microculture plates (2×10⁵ cells/well) for 72 hours, at 37° C. in humidified air containing 7.5% CO₂. Each well was pulsed with 2 μCi of [³H]-Thymidine (specific activity 10 Ci/mmol) for the final six hours. The cultures were then harvested on fiberglass filters and the proliferative response expressed as CPM±S.E. or as stimulation index (SI) (mean CPM of test cultures divided by mean CPM of control cultures).

Reverse transcriptase polymerase chain reaction (RT-PCR) analysis: RT-PCR analysis, verified by Southern blotting, was utilized on brain samples according to the protocol described elsewhere with some modifications (39). Rats were euthanized by CO₂ narcosis. Brain samples containing mainly the midbrain and brain stem were obtained after perfusion of the rat with 160-180 ml of ice-cold phosphate buffered saline (PBS) injected into the left ventricle following an incision in the right atrium. Each sample was homogenized. Total RNA was extracted using the Tri-Zol procedure (Gibco BRIE) according to the manufacturer's protocol. mRNA was then isolated using a MRNA isolation kit (#1741985, Boheringer Mannheim, Germany), and reverse transcribed into first strand cDNA as described in detail elsewhere (39). First strand cDNA was then subjected to 35 cycles of PCR amplification using specific oligonucleotide priers to rat IGIF and IFN-γ which were designed based on the published sequence of each cytokine (NCBI accession number for rat IGIF—U77777; and for rat IFN-γ—M29315) as follows (Table 1):

TABLE 1

| | | SEQ |
|---|---|---|
| Name/Function | Sequence | ID NO: |
| Rat IGIF | 5′-ATGGCTGCCATGTCAGAAGAAG-3′ | 2 |
| sense | | |
| Rat IGIF | 5′-CTAACTTTGATGTAAGTTAGTAAGA-3′ | 3 |
| antisense | | |
| Rat IFN-γ | 5′-TACTGCCAAGGCACACTCATTGAA-3′ | 4 |
| sense | | |
| Rat IFN-γ | 5′-CGCTTCCTTAGGCTAGATTCTGG-3′ | 5 |
| antisense | | |
| Rat β-actin | 5′-CATCGTGGGCCGCTCTAGGCA-3′* | 6 |
| sense | | |
| Rat β-actin | 5′-CCGGCCAGCCAAGTCCAGACG-3′* | 7 |
| antisense | | |

Sequence acccording to Reference 39.

Experimental conditions were calibrated so RT-PCR amplifications fall on the linear part of the titration curve. The cycle profile was: denaturation at 95° C. for 60 seconds, annealing at 55° C. for 60 seconds, and elongation at 72° C. for 60 seconds. Amplified products were subjected to electrophoresis, transferred to a nylon membranes (MagnaGraph nylon transfer membrane, msi, Westborough, Mass.), fixed with ultraviolet light (120 mjoules) and hybridized with 10⁶ cpm/ml of a ³²P labeled DNA fragments encoding the fill length PCR product of IGIF and of β-actin (random priming: Amersham, Arlington Heights, Ill.). PCR products were used as probes only after each PCR product was cloned and its sequence was verified as described below. Southern bolt images were objectively assessed using an FujiFilm Thermal System FTI-500 (FujiFilm, Japan).

Cloning and sequencing of PCR products: Each of the amplified PCR products described above was cloned into a pUC57/T vector (T-cloning Kit #K1212, MBI Fermentas, Lithuania) and transformed to E. coli according to the manufacturer's protocol. Each clone was then sequenced (Sequenase version 2, USB, Cleveland, Ohio) according to the manufacturer's protocol.

Production and purification of recombinant proteins. After sequence verification, PCR products were recloned into a PQE expression vector (PQE-30, PQE-31 or PQE-32 according the correct open reading frame) and expressed in E Coli (Qaigen, Hilden, GmbH) and then purified by an NI-NTA-super flow affinity purification of 6× His proteins (Qaigen). Each recombinant protein sequence has been verified (N -terminus).

Producdon and purification of Rabbit and-rat IGIF IgG. Rabbit anti-rat IGIF antibodies were generated as described (40) and IgO fraction was purified using a MiTrap protein G kit (Pharmacia, Piscataway, N.J., Kit #17-040-01). Antibody titer was determined by a direct ELISA assay; ELISA plates (Nunc, Denmark) were coated with recombinant rat IGIF(50 ng/well). Rabbit anti-rat IGIF (IgG fraction) was added in seuial dilutions from 2⁸ to 2³⁰. Goat anti-rabbit lgG alkaline phosphatase conjugated antibodies (Sigma) were used as a labeled antibody. p-Nitrophenyl Phosphate(p-NPP) (Sigma) was used as a soluble alkaline phosphatase substrate. Results of triplicates wire calculated as log 2 antibody titer±SE. The purified anti-rat IGIF IgG titer was 18±0.4.

Cytokine determination: Spleen cells from EAE donors were stimulated in vitro (10⁷ cells/mi) in 24 well plates-(Nunc) with 100 ILM p68-86. Spleen cells from naive donors were cultured (10⁷ cells/ml, 24 well plates) with 2 μg/ml Con A (Sigma). After 72 hours of stimulation, supernatants were assayed by semi-ELISA kits, that include antibody pairs and recombinant rat cytokines, as follows: IFN-γ, rabbit anti-rat IFN-γ polyclonal antibody (CY-048, Innogenetics, Belgium) as a capture antibody, biotinylated mouse anti-rat monoclonal antibody (CY-106 clone BD-1, Innogenetics) as a detection antibody, and Alkaline phosphatase-Streptavidin (cat No. 43-4322, Zymed, SF, CA) with rat recombinant IFN-γ as a standard (Cat No 3281SA, Gibco BRL); TNF-α, commercial semi-ELISA kit for the detection of rat TNF-α, (Cat No 80-3807-00, Genzyme, Cambridge, Mass.); IL-4, mouse anti-rat IL-4 monoclonal antibody (24050D OX-81, PharMingen, San Diego, Calif.) as a capture antibody, and rabbit anti-rat IL-4 biotin-conjugated polyclonal antibody (2411-2D, PharMingen) as second antibody. Recombinant rat IL-4 purchased from R&D (504-RL) was used as a standard.

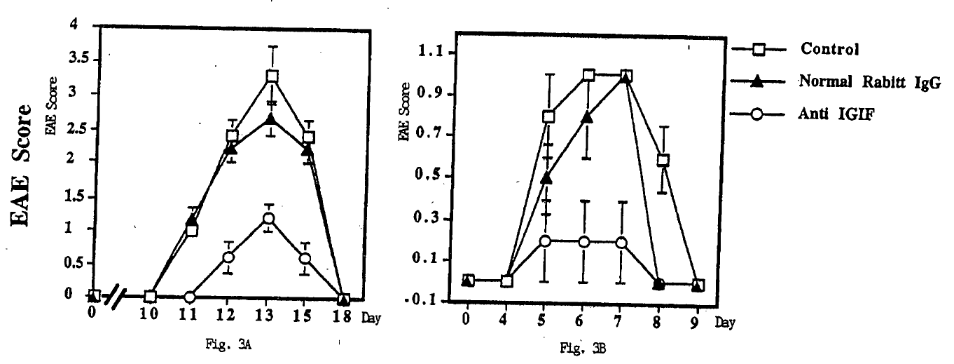

Statuvdcal analysis: Significance of differences was examined using Student's t-test. Mann-Whitney sum of ranks test was used to evaluate significance of differences in mean of maximal clinical score (FIG. 3). Value of p<0.05 was considered significant.

### Experimental Results

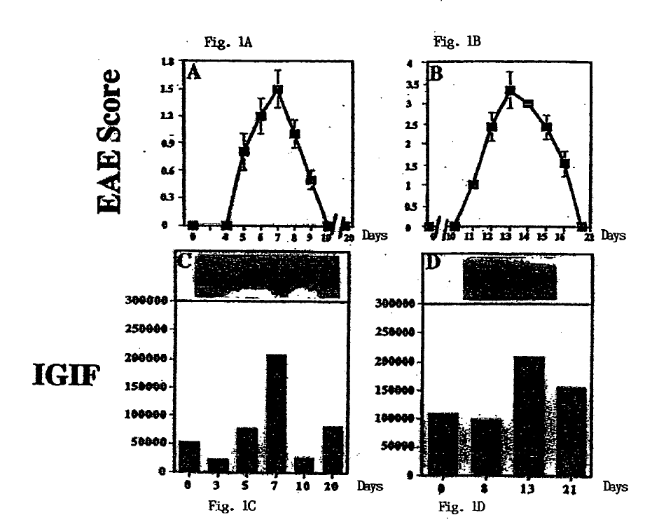

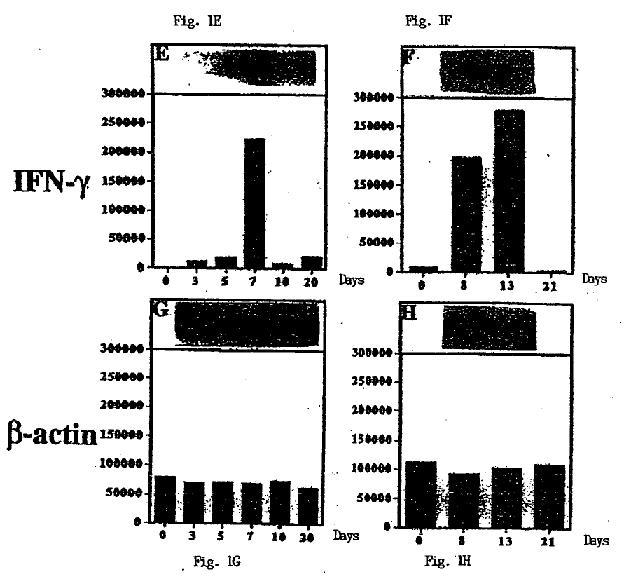

IGIF mRNA is transcribed in the inflamed EAE brain: Midbrain-brain stem samples were obtained from rats with developing transferred EAE (FIG. 1A) before adoptive transfer of disease (day 0), and at various time points: before the onset of disease (day 3), at the day of onset (day 5), the peak (day 7), following recovery (day 10), and 10 days after recovery (day 20). For each time point, samples from six different brains were. subject to RT-PCR analysis using specific oligonucleotide primers which constructed to IGIF and IFN-γ. Each amplification was calibrated to α-actin and verified by Southern blot analysis. This enabled semi-quantitative analysis of the dynamics of mRNA transcription of IGIF and IFN-γ at the site of inflammation. FIGS. 1C and 1E show representative results from each time point of the experiment. A substantial increase in the transcription of both IGIF and IFN-γ mRNA in EAE brains was observed at the peak of disease (day 7). The augmented transcription of IFN-γ MRNA reverted to background levels during recovery. Unexpectedly, a notable transcription of IGIF MRNA could be observed even ten days after recovery (FIG. 1C).

Rats with developing active disease manifested similar MRNA transcription characteristics as those with developing transferred disease. That is, a substantial increase in the transcription of both IGIF and IFN-γ MIRNA in EAE brains was observed at the peak of disease (day 13). The augmented transcription of IFN-γ, but not of IGIF mRNA, regressed to background level during recovery (FIGS. 1D and 1F). A substantial increase in the level of a IGIF transcription at the site of inflammation in the CNS during the course of disease may suggest involvement in its regulation. To evaluate this point, the role of IGIF in regulation of EAE was investigated.

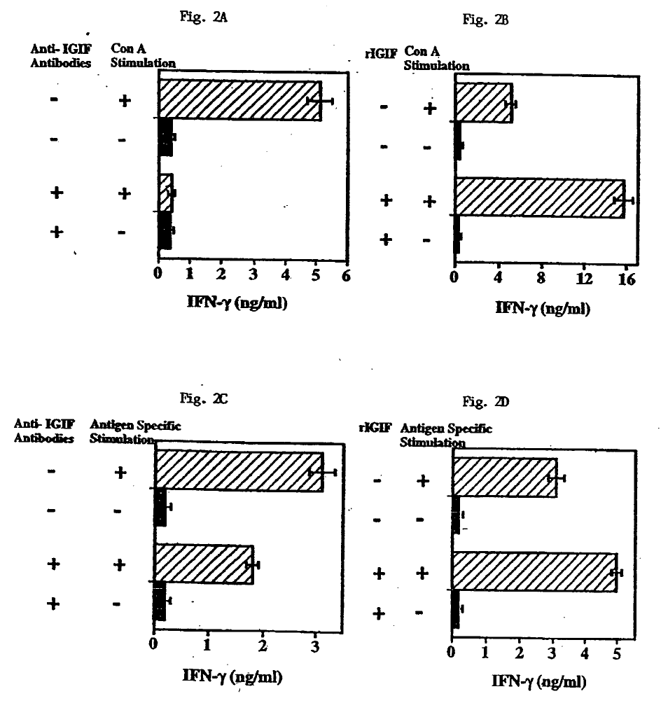

Recombinant rat IGIF and its neutralizing antibodies affect IFN-γ production by activated Tcells from naive donors more significantly than by antigen specific primed T-cells: PCR products encoding rat IGIF were used to generate the recombinant protein which was used to produce anti-IGIF neutralizing antibodies. These antibodies .sgnificantly reduced the production of IFN-γ in primed T-cells proliferating in response to their specific myelin basic protein (MBP) epitope (FIG. 2C 3.2±0.25 versus 1.8±0.11 ng/ml with backgrounds of 0.2±0.1 and 0.25±0.1, p<0.01) and entirely blocked IFN-γ production in Con A activated T-cells from naive donors (FIG. 2A, 5.1±0.4 versus 0.4 2±0.1 ng/ml with backgrounds of 0.4±0.1 and 0.36, p<0.001). Control IgG from normal rabbit serum did not exert a notable effect on IFN-γ production by either Con A activated naive spleen cells or MBP p68-86 primed spleen cells (data not shown). Recombinant rat IGIF elicited IFN-γ production in Con A activated splenic T-cells from naive donors (FIG. 2B, 15.8±0.8 ng/ml versus 5.1±0.3 with backgrounds of 0.3±0.1 and 0.4±0.15, p<0.001) and significantly, though again less profoundly, the response of primed spleen T-cells to their target MBP antigen (FIG. 2D, 4.97±0.15 ng/ml versus 3.2±0.25, with backgrounds of 0.3±0.15 and 0.25±0.1, p<0.001). Thus, both the inhibitory effect of IGIF neutralizing antibodies and the augmentation by IGIF of IFN-γ production are more profound on activated T-cells from a naive donor than on printed T-cells responding to their target epitope. It has recently been suggested that IGIF primarily affects IFN-γ production by Th1 not Th2 cells (29). It is possible that immunization with p68-86/CFA induces a substantial selection of antigen specific Th2 cells, albeit not enough to inhibit the subsequent development of a Th1 mediated autoimmune disease.

The in vitro addition of either anti-IGIF antibodies or of recombinant IGIF did not affect the antigen specific proliferative response developed in primed 4splenic T-cells responding to MBP p68-86 (SI=4.2±0.3, 3.6±0.4 and 3.9±0.3 in control spleen T-cells) versus cultured spleen cells supplemented with either anti-IGIF antibodies or recombinant IGIF.

Neutralizing antibodies to recombinant rat IGIF block the development of both active and travferred EAE: The role of anti-IGIF antibodies in the regulation of T-cell mediated autoimmune diseases has never been explored before. Herein the competence of the anti-IGIF neutralizing antibodies to inhibit active (FIG. 3A) and transferred (FIG. 3B) EAE is evaluated. Lewis rats were immunized with p⁶⁸-86 /CFA to develop active EAE. Just before the onset of disease (days eight and ten) and at the onset of disease (day eleven) these rats were injected with either rabbit anti-rat IGIF (IgG fraction), IgG fraction purified from non-immunized rabbits (control IgG), or with PBS, and monitored for clinical signs of EAE. Control PBS treated rats and rats treated with control ISO all (6/6 rats in each group) developed severe EAE (Mean maximal clinical score 3.3±0.43 and 2.66±0.26, respectively). In contrast, rats treated with anti-IGIF antibodies developed a markedly reduced disease (FIG. 3A, incidence 5/6, mean maximal clinical score 1.2±0.2, p<0.01).

The competence of anti-IGIF antibodies to inhibit transferred EAE (FIG. 3B) was further evaluated. Three, five and seven days after adoptive transfer of disease rats were injected as described above and monitored for clinical signs of EAE. While control PBS treated rats and rats treated with control IgG. have all (6/6 rats in each group) developed EAE (mean maximal clinical score 1±0 in each group) rats administered with anti-IGIF antibodies were highly protected (FIG. 3B, incidence 1/6, mean maximal clinical score 0.2±0.1, p<0.01) Thus, immunotherapy with anti-IGIF serves as a powerful tool to block the development of actively induced or transferred EAE.

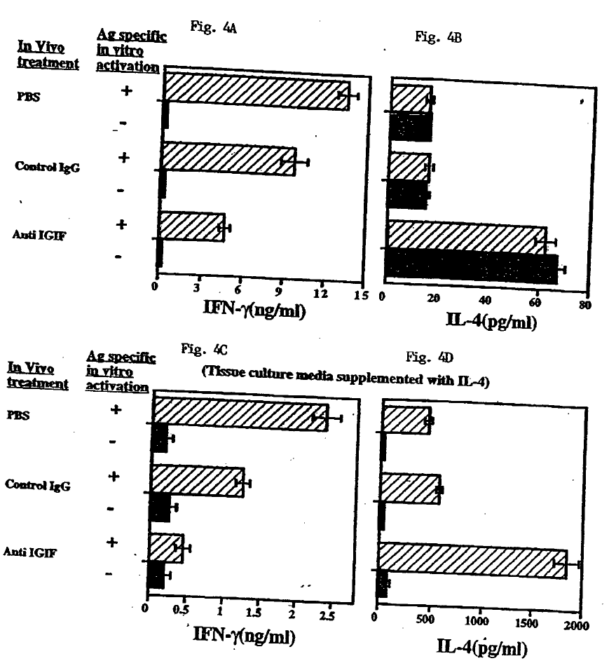

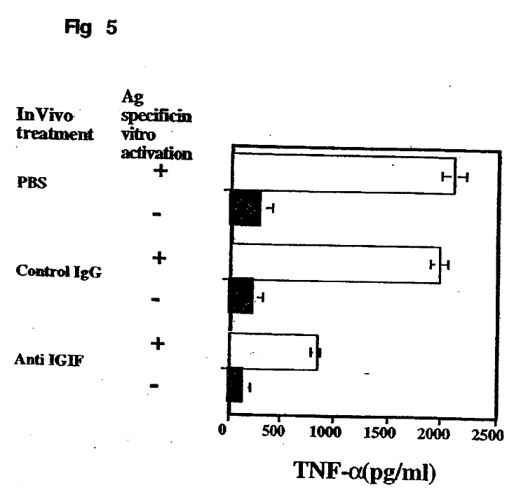

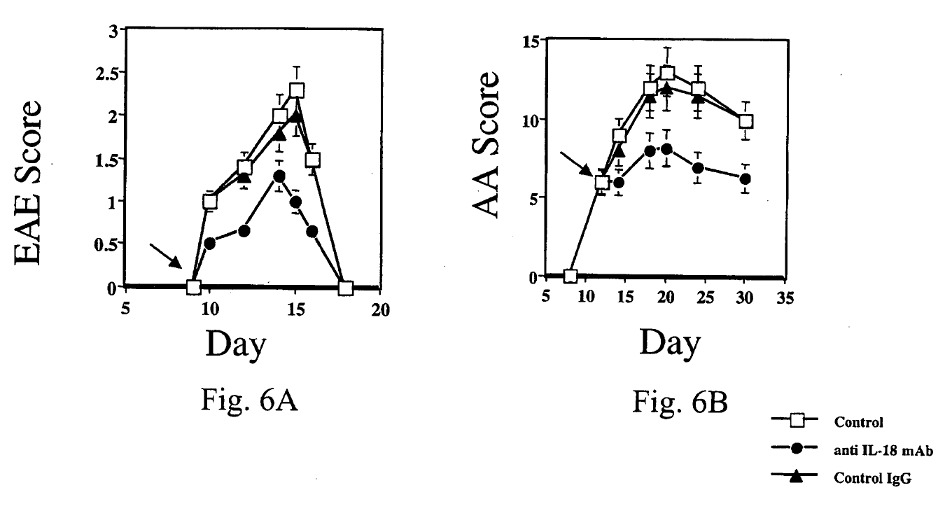

Alteraton of IFN-γ and IL-4 production in EAE rats injected with anti-IGIF neutralizing antibodies suggests that perturbation of the Th2/Th1 balance contributes to disease blockade: The possible involvement of a Th2/Th1 switch in EAE inhibition by anti-IGIF immunotherapy has been evaluated (FIG. 4). Lewis rats were immunized with p68-86/CFA to develop active EAE. Five and seven days later these rats were injected with either PBS, control rabbit IgG or rabbit anti-rat IGIF (IgG fraction). Two days after the last treatment, splenic T-cells were cultured with MBP p68-86 in stimulation medium that was (FIGS. 4C and 4D) or was not supplemented with recombinant rat IL-4 (FIGS. 4A and 4B). In spleen cells cultured form MBP 68-86 primed donors, IFN-γ was produced only when the priming antigen was added to the culture (FIGS. 4A, 0.3±0.1 ng/ml without addition of MBP 68-86 versus 13.5±0.7 in cells proliferating to p68-86). Addition of recombinant IL-4 led to a significant decrease in IFN-γ which was still dependent upon antigenic stimulation (FIGS. 4A and 4C 0.19±0.08 ng/ml without addition of MBP 68-86 versus 2.37±0.8 in cells proliferating to p68-86, a 12 fold increase). Spleen T-cells from anti-IGIF treated rats produced markedly reduced levels of IFN-γ in response to antigenic stimulation in cultures that were or were not supplemented with IL-4 (FIG. 4A, 4.7±0.4 ng/ml in spleen cells from anti-IGIF treated rat versus 9.7±0.8 in spleen cells from rats treated with normal rabbit IgG and 13.5±0.7 in PBS treated rats, with backgrounds of 0.4, 0.8 and 0.7, p<0.001, when comparing anti-IGIF treatment to each control group). IL-4 production, however, markedly increased in splenic T-cells from anti-IGIF treated rats regardless of in vitro stimulation (FIG. 4B, 62.3±4.2 pg/ml in spleen cells from anti-IGIF treated rat versus 15.3±0.4 in splcen cells from rats treated with normal rabbit IgO and 15.6±0.6 in PBS treated rats , p<0.001, when comparing anti-IGIF treatment to each control group) unless cultures were supplemented with IL-4 (FIG. 4D, 1860±120 pg/ml in spleen cells from anti-IGIF treated-rat versus 570±30 in spleen cells from rats treated with normal rabbit IgG and 450±35 in PBS treated rats, with backgrounds of 85, 42 and 34, p<0.0001, when comparing anti-IGIF treatment to each control group). Addition of IL-4 to cultured spleen T-cells (FIG. 4C-D) did not exhibit a notable effect on their antigen specific proliferative response (data not shown).

TNF-α production was then evaluated in spleen cells form the above groups. The above spleen cells from anti-IGIF treated rats produced markedly reduced levels of TNF-α in response to antigenic stimulation (FIG. 5, 850±45 pg/ml in spleen cells from anti-IGIF treated rats versus 1975±80 in spleen cells from rats treated with normal rabbit IgG and 2100±110 in PBS treated rats, with backgrounds of 230, 210 and 270, respectively, p<0.001, when comparing anti-IGIF treatment to each control group). Thus perturbation of the Th1/Th2 balance in anti-IGIF treated rats is associated with a marked reduction in TNF-α production.

Finally, the proliferative response of each group of cultured spleen cells to p68-86 was evaluated in a proliferation assay. (SI=4.2±0.3, 3.6±0.4 and 3.96±0.5 in spleen cells from rats treated with either anti-IGIF, normal rabbit IgG or PBS respectively). Thus anti-IGIF immunotherapy alters Th2/Th1 balance without a notable affect on antigen specific proliferative responsiveness.

### Example 2

### Preparation and Characterization of an and IL-18 Mab

To produce monoclonal antibodies against IL-18, recombinant IL-18 produced and purified as previously described (Wildbaum et al, J. Immunol. 1998) was administered to BALB/C (50 μg IL-18 in 200 μl CFA, i.p.). Three and six and nine weeks later these mice were subjected to repeated administration of 50 μg IL-18 in 200 μl IFA, i.p. Eight days later development of anti IL-18 antibodies was verified in sera of each minute (ELISA); spleens were then removed for mAb production. Screening for positive mAb was effected by determining the ability of the Mabs to bind recombinant IL-18, but not IL-1β (ELISA), and their ability to shift antigen specific T-cell polarization towards Th2 in an in vitro system (Wildbaum et al, J Immunol, 1998). Based on this screening system clone 7B3 was selected for in vivo experimental work.

FIG. 6A illustrates the ability of mAb clone 7B3 (IgG1) to suppress ongoing EAE. A group of 18 rats was subjected to active induction of EAE. At the onset of disease these rats were sub-grouped to 3 groups of 6 rats each and subjected to repeated administration (every other day) of 500 μg/ml of mAb 7B3, or isotype matched control mouse IgG1 (Sigma). Clinical scores was monitored daily. The experimental methods of disease induction and monitoring are explained in details elsewhere (Wildbaum et al, J Immunol, 1998). Results are shown as mean maximal score ±SE. Only rats administered with mAb 7B3 exhibited a significantly (p<0.05) reduced form of the disease.

FIG. 6B illustrates the ability of mAb 7B3 (IgG1) to suppress ongoing adjuvant arthritis (AA). A group of 18 rats was subjected to active induction of AA. At the onset of disease these rats were sub-grouped to 3 groups of 6 rats each and subjected to repeated administration (every other day) of 500 μg/ml of mAb 7B3, or isotype matched control mouse IgG1 (Sigma). Clinical scores was monitored daily. The experimental methods of disease induction and monitoring are explained in detail elsewhere (Wildbaum, Youssef & Karir, J. Immunol, 2000). Results are shown as mean maximal score

SE. Only rats administered with mAb 7B3 exhibited a significantly (p<0.05) reduced form of disease.

These results along with previously published findings that show that Th1 cells which produce interferon gamma (IFN-γ) and TNF-α elicit a pro-inflammatory process in various T-cell mediated autoimmune diseases and that T-cell polarization from Th1 to Th2 suppresses disease formation, clearly indicate that the present invention can be used to treat a wide range of diseases.

Other than multiple sclerosis and rheumatoid arthritis, the present invention can also be utilized to treat type I diabetes, uveoretinitis and intestinal bowel disease (Crohn's disease and ulcerative colitis) and other related diseases (Ando et al., 1989; Cash et al., 1994; Healey et al., 1995; Katz et al., 1995; Khoruts et al., 1995; Kuchroo et al., 1995; Leonard et al., 1995; Racke et al., 1994; Rapoport et al., 1993; Rott et al., 1994; Saoudi et al., 1993; Seder et al., 1993; Trinchieri, 1993; Yang et al., 1994).

Thus, the present study and data accumulated from prior art studies clearly indicate that IGIF neutralization via use of, for example, a monoclonal antibody can be utilized to treat a wide range of T-cell mediated autoimmune diseases.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fail within the spinet and broad scope of the appended claims. All publications, patents and patent applications and GenBank Accession numbers mentioned in this specification are herein incorporated in their entirety by reference into the specification, to the same extent as if each individual publication, patent or patent application or GenBank Accession number was specifically and individually indicated to be incorporated herein by reference. In addition, citation or identification of any reference in this application shall not be construed as an admission that such reference is available as prior art to the present invention.

### REFERENCES CITED

1. O'Garra, A., and K. Murphy. 1994. Role of cytokines in determining T-lymphocyte function. Curr Opin Immunol 6:458-66.

2. Abbas, A. K., K. M. Murphy, and A. Sher. 1996. Functional diversity of helper T lymphocytes. Nature 383:787-93.

3. Swain, S. L., A. D. Weinberg, M. English, and G. Huston. 1990. IL-4 directs the development of Th2-like helper effectors. J Immunol 145:3796-3806.

4. Lederer, J. A., V. L. Perez, L DesRoches, S. M. Kim, A. K. Abbas, and A. H. Lichtman. 1996. Cytokine transcriptional events during helper T-cell subset differentiation. J Exp Med 184:397-406.

5. Seder, R. A., R. Gazzinelli, A. Sher, and W. F. Paul 1993. IL-12 acts directly on CD4⁺ T-cells to enhance priming for IFN-□ production and diminishes IL-4 inhibition of such priming. Proc. Natl. Acad. Sci. USA. 90:10188-10192.

6. Seder, R. A., W. E. Paul, M. M. Davis, and S. Fazekas de St. Groth. 1992. The presence of interleukin4 during in vitro priming determines the cytokine-producing potential of CD4⁺ T-cells from T-cell receptor transgenic mice. J. Exp. Med. 176:1091-1098.

7. Fiorentino, D. F., A. Zlotnik, P. Viefra, T. R. Mosmann, M. Howard, K. W. Moore, and A. O'garra. 1991. IL-10 acts on the antigen presenting cell to inhibit cytokine production by Th1 cells. J. Immunol. 146:3444-3451.

8. Mosmann, T. R., and R. L. Coffman. 1989. Th1 and Th2 cells: different patterns of lymphokine secretion lead to different functional properties. Annue.Rev.lmmunol. 9:145-173.

9. Mosmann, T., and K. Moor. 1989. The role of IL-10 in the crossregulation of Th1 and Th2 responses. Immunol. Today. 12. A49-A53.

10. Huang, H., J. Hu-Li, H. Chen, S. Z. Ben-Sasson, and W. E. Paul. 1997. IL-4 and IL-13 production in differentiated T helper type 2 cells is not IL-4 dependent J Immunol 159-3731-8.

11. Fukaura, H., S. C. Kent, M. J. Pietmisewicz, S. J. Khoury, H. L. Weiner, and D. A. Hafler. 1996. Induction of circulating myelin basic protein and proteolipid protein-specific transforming growth factor-betal-secreting Th3 T-cells by oral administration of myelin in multiple sclerosis patients. Journal of Clinical Investigation 98:70-7.

12. Racke, M. K., A Bonomo, D. E. Scott, B. Cannella, A. Levine, C. S. Raine, E. M. Shevach, and M. Rocken. 1994. Cytokine-induced immune deviation as a therapy for inflammatory autoimmune disease. JExp Med 180:1961-6.

13. Cash, E., A. Minty, P. Ferrara, D Caput, D. Fradelizi, and O. Rott. 1994. Macrophage-inactivating IL-13 suppresses experimental autoimmune encephalomyelitis in rats. J Immunol 153:4258-67.

14. Kuchroo, V. K., M. P. Das, J. A. Brown, A. M. Ranger, S. S. Zamvil, R. A. Sobel, H. L. Weiner, N. Nabavi, and L. H. Glimeher. 1995. B7-1 and B7-2 costimulatoxy molecules activate differentially the Th1/Th2 developmental pathways: application to autoimmune disease therapy. Cell 80:707-18.

15. Leonard, J. P., K. E. Waldburger, and S. J. Goldman. 1995. Prevention of experimental autoimmune encephalomyclitis by antibodies against interleukin 12. J Exp Med 181:381-6.

16. Healey, D., P. Ozegbe, S. Arden, P. Chandler, J. Hutton, and A. Cooke. 1995. In vivo activity and in vitro specificity of CD4⁺ Th1 and Th2 cells derived from the spleens of diabetic NOD mice. J Clin Invest 95: 2979-85.

17. Khoruts, A, S. D. Miller, and M. K. Jenkins. 1995. Neuroantigen-specific Th2 cells are inefficient suppressors of experimental autoimmune encepbalomyelitis induced by effector Th1 cells. Journal of Immunology 155:5011-7.

18. Ando, D. G., J. Clayton, D. Kono, J. L. Urban, and E. E. Sercarz. 1989. Encephalitogenic T-cells in the B10.PL model of experimental allergic encephalomyelitis (EAE) are of the Th-1 lymphokine subtype. Cell Immunol 124:132-43.

19. Katz, J. D., C. Benoist, and D. Mathis. 1995. T helper subsets in insulin dependent diabetes. Science 268:1185-1188.

20. Liblau, R. S., S. M. Singer, and H. O. McDevitt. 1994. Th1 and Th2 CD4+ T-Cells in the pathogenesis of organ-specific autoimmune diseases. Immunol. Today in press.

21. Saoudi, A., J. Kuhn, K. Huygen, K. Y. de, T. Velu, M. Goldman, P. Druet, and B. Bellon. 1993. TH2 activated cells prevent experimental autoimmune uveoreinitis, a TH1-dependent autoimmune disease. Eur J Immunol 23 3096-103.

22. Rapoport, M. J., A. Jaramillo, D. Zipris, A. Lazarus, D. V. Serreze, E. H. Leiter, P. Cyopick, J. S. Danska, and T. L. Delovitch. 1993. Interleukin-4 reverses T-cell proliferative unresponsiveness and prevents the onset of diabetes in nonobese diabetic mice. J. Exp. Med. 178:87-99.

23. Rott, O., B. Fleischer, and E. Cash. 1994. Interleukin-10 prevents experimental allergic encephalomnyelitis in rats. Eur J Immunol 24:1434-40.

24. Trinchieri, G. 1993. Interleukin-12 and its role in the generation of Th1 cells. Immunol, Today 14:335-337.

25. Okamura, H., H. Tsutsi, T. Komatsu, M. Yutsudo, A. Hakura, T. Tanimoto, K. Torigoe, T. Okura, Y. Nukada, K. Hattori, and et al. 1995. Cloning of a new cytokine that induces IFN-gamma production by T-cells [see comments]. Nature 378:88-91.

26. Bazan, J. F., J. C. Timas, and R. A. Kastelein. 1996. A newly defined interleukin 1? Nature 379:591.

27. Ghayur, T., S. Banedee, M. Huin, D. Butler, L. Herzog, A. Carter, L. Quintal, L. Sekut, R. Talanian, M. Paskind, W. Wong, R. Kamen, D. Tracey, and Hi. Allen. 1997. Caspase-1 processes IFN-gamma-inducing factor and regulates LPS-induced IFN-gamma production. Nature 386:619-23.

28. Gu, Y., K. Kuida, H. Tsutsui, G. Ku, K. Hsiao, M. A. Fleming, N. Hayashi, K. Higashino, H. Okamura, K. Nakanisbi, M. Kurimoto, T. Tanimoto, R. A. Flavell, V. Sate, M. W. Harding, D J. Livingston, and M. S. Su. 1997. Activation of interferon-gamma inducing factor mediated by interleukin-1beta converting enzyme. Science 275:206-9.

29. Robinson, D., K Shibuya, A. Mui, F. Zonin, E. Murphy, T. Sana, S. B. Hartley, S. Menon, R. Kastelein, F. Bazan, and A. O'Garra. 1997. IGIF does not drive Th1 development but synergizes with IL-12 for interferon-gamma production and activates IRAK and NFkappaB. Immunity 7:571-81.

30. Rothe, H., T. Hibino, Y. Itoh, H. Kolb, and S. Martin. 1997. Systemic production of interferon-gamnmra inducing factor (IGIF) versus local IFN-gamma expression involved in the development of Th1 insulitis in NOD mice. J Autoimmun 10:251-6.

31. Micallef, M. J., T. Ohtsuki, K. Kohno, F. Tanabe, S. Ushio, M. Namba, T. Tanimoto, K Torigoe, M. Fujii, M. Ikeda, S. Fukuda, and M. Kurimoto. 1996. interferon-gwmma-inducing factor enhances T helper 1 cytokine production by stimulated human T-cells: synergism with interleukin-12 for interferon-gamma production. European Journal of Immunology 26:1647-51.

32. Ahm, H. J., S. Maruo, M. Tomura, J. Mu, T. Hamaoka, K Nakanishi, S. Clark, M. Kurimoto, H. Okamura, and H. Fujiwara. 1997. A mechanism underlying synergy between IL-12 and IFN-gamma-inducing factor in enhanced production of IFN-gamma. J Immunol 159:2125-31.

33. MacFarlin, D., and H. MacFarIand. 1983. Multiple Sclerosis N. Engl. J. Med. 307:1183-1188.

34. Alvord, E. C. J., M W. Kies, and A. J. Suckling. 1984. Experimental Allergic Encephalomylitis: A useful Model for Multiple Sclerosis. In Progress in clinical and biological research., vol. 146. E. C. J. Alvord, M. W. Kies, and A. J. Suckling, eds. Allen R. Liss, New York, p. 1-537.

35. Rainc, C. S. 1991. Multiple sclerosis; a pivotal role for the T-cell in lesion development. Neuropathol Appl Neurobiol 17:265-74.

36. Oksenberg, J. R., M. A. Panzara, A. B. Begovich, D. Mitchell, H. A. Erlich, R. S. Murray, R. Shimonkevitz, M. Sherritt, J. Rothbard, C C. Bernard, and a. 1. et. 1993. Selection for T-cell receptor V beta-D beta-J beta gene rearrangemnents with specificity for a myelin basic protein peptide in brain lesions of multiple sclerosis. Nature 362:68-70.

37. Ben-Nun, A., H. Wek-erle, and L. R. Cohen. 1981. The rapid isolation of clonable antigen-specific T lymphocyte lines capable of mediating autoimmune encephalomyelitis. Eur J Immunol 11:195-9.

38. Brocke, S., K. Gijbels, M. Allegretta, M. Ferber, C. Piercy, T. Blankenstein, R. Martin, U. Utz, N. Karin, D. Mitchell, and et al. 1996. Treatment of experimental encephalomyelitis with a peptide analogue of myelin basic protein. Nature 379:343-6.

39. Karin, N., F. Szafer, D. Mitchell, D. P. Gold, and L. Steimnan. 1993. Selective and nonselective stages in homing of T lymphocytes to the central nervous system during experimental allergic encephalomyelitis. J Immunol 150:4116-24.

40. Harlow, E., and D. Lane. 1988. Antibodies a laboratory manual. Cold Spring Harbor Laboratory, New York.

41. Waisbman, A., P. J. Ruiz, D. L. Hirschberg, A. Gelman, J. R. Oksenberg, S. Brocke, F. Mor, I. R. Cohen, and L. Steinman. 1996. Suppressive vaccination withl DNA encoding a variable region gene of the T-cell receptor prevents autoimmune encephalomyelitis and activates Th2 immunity. Nature Medicine 2:899-905.

42. Ramierz, F., D. J. Fowell, M. Puklavec, S. Simmonds, and D. Mason. 1996. Glucocorticoids promote a TH2 cytoldne response by CD4+ T-cells in vitro. J Immunol156:2406-12.

43. Saoudi, A., S. Simmonds, I. Huitinga, and D. Mason. 1995. Prevention of experimental allergic encephalomyclitis in rats by targeting autoantigen to B cells: evidence that the protective mechanism depends on changes in the cytokine response and migratory properties of the autoantigen-specific T-cells [see comments]. J Exp Med 182:335-44.

44. Thornhill, M. H., S. M. Wellicome, D. L. Mahiouz, J. S. Lanchbury, A. U. Kyan, and D. O. Haskard. 1991. Tumor necrosis factor combines with IL-4 or IFN-gamma to selectively enhance endothelial cell adhesiveness for T-cells. The contribution of vascular cell adhesion molecule-1-dependent and independent binding mechanisms. J Immunol 146:592-8.

45. Baron, J. L., J. A. Madri, N. H. Ruddle, G. Hashim, and C. J. Janeway. 1993. Surface expression of alpha 4 integrin by CD4 T-cells is required for their entry into brain parenchyma. J Exp Med 177:57-68.

46. Yednock, T. A., C. Cannon, L. C. Fritz, M. F. Sanchez, L. Steinman, and N. Karin. 1992. Prevention of experimental autoimmune encephalomyelitis by antibodies against alpha 4 beta 1 integrin. Nature 356:63-6.

47. Kuroda, Y., and Y. Shimarnoto. 1991. Human tumor necrosis factor-alpha augments experimental allergic encephalomyelitis in rats J Neuroimmunol 34:159-64.

48. Powell, M. B., D. Mitchell, J. Lederman, J. Buckneier, S. S. Zamvil, M. Graham, N. H. Ruddle, and L. Steinman. 1990. Lymphotoxin and tumor necrosis factor-alpha production by myelin basic protein-specific T-cell clones correlates with encephalitogenicity. Int Immunol 2:539-44.

49. Ruddle, N. H., C. M. Bergman, K. M. McGrath, E. G. Lingenheld, M. L. Grunnet, S. J. Padula, and R. B. Clark. 1990. An antibody to lymphotoxin and tumor necrosis factor prevents transfer of experimental allergic encephalomyelitis. J Exp Med 172:1193-200.

50. Selmaj, K., C. S. Raine, and A. H. Cross. 1991. Anti-tumor necrosis factor therapy abrogates autoimmune demyelination. Ann Neurol 30:694-700.

51. Kohno, K., J. Kataoka, T. Ohtsuki, Y. Suemoto, T. Okamoto, M. Usui, M. Ikeda, and M. Kurimoto. 1997. IFN-gamma-inducing factor (IGIF) is a costimulatory factor on the activation of Th1 but not Th2 cells and exerts its effect independently of IL-12. J Immunol 158:1541-50.

52. Rothe, H., N. A. Jenkins, N. G. Copeland, and H. Kolb. 1997. Active stage of autoimmune diabetes is associated with the expression of a novel cytokine, IGIF, which is located near Idd2. J Clin Invest 99:469-74.

53. Karin, N, J. D. Mitchell, S. Brocke, N. Ling, and L. Steinman. 1994. Reversal of experimental autoimmune encephalomyelitis by as soluble peptide variant of a myelin basic protein epitope: T-cell receptor antagonism and reduction of IFN-g and TNF-a production. J Exp. Med, 180:2227-2237.

54. Grewal, I. S., H. G. Foellmer, K. D. Grewal, J. Xu, F. Hardardottir, J. L. Baron, C. A. Janeway, Jr., and R. A. Flavell. 1996. Requirement for CD40 ligand in costimulation induction, T-cell activation, and experimental allergic encephalomyelitis. Science 273:1864-7.

55. Krakowski, M., and T. Owens. 1996. Interferon-gamma confers resistance to experimental allergic encephalomyelitis. Eur J Immunol 26:1641-6.

56. Heremans, H., C. Dillen, M. Groenen, E. Martens, and A. Billiau. 1996. Chronic relapsing experimental autoimmune encephalomyelitis (CREAE) in mice: enhancement by monoclonal antibodies against interferon-gamma, Eur J Immunol 26:2393-8.

57. Billiau, A., H. Heremans, F. Vandekerckhove, R. Dijlonans, H. Sobis, E. Mealepas, and H. Carton. 1988. Enhancement of experimental allergic encephalomyelitis in mice by antibodies against IFN-gamma. J Immunol 140:1506-10.

58. Segal, B. M., B. K. Dwyer, and E. M. Shevach. 1998. An interleukin (IL)-10/IL-12 immunoregulatory circuit controls susceptibility to autoimmune disease. J Exp Med 187:537-46.

59. Wildbaum G, Youssef S, Grabie N, Karin N. Neutralizing antibodies to IFN-gamma-inducing factor prevent experimental autoimmune encephalomyelitis. J Immunol 1998;161(11):6368-74.

60. Wildbaum G, Youssef S, Kann N. A targeted DNA vaccine augments the natural immune response to self TNF-alpha and suppresses ongoing adjuvant artitis. J Immunol 2000;165(10):5860-6.

61. Rotnagnani, S., Parronchi, P., D'Elios, M., Romagnani, P., Annunziato, F., Menghetti, M., Maggi, E., and Del Prete, G. (1996). Immune mechanisms in the pathogenesis of inflammatory gastrointestinal disorders. Ital J Gastroenterol 28, 11-17.

62. Yang, X.-D., Tisch, R., Singer, S. M., Cao, Z. A, Liblau, R., Schreiber, R. D., and McDevitt, H. O. (1994). Effect of tumor necrosis factor a on insulin-dependent diabetes; mellitus in NOD mice. I. The early development of autoimmunity and diabetogenic process. J Exp Med 180, 995-1004.

What is claimed is:

1. An antibody or antibody fragment capable of specifically neutralizing an interferon gamma inducing factor.

2. The antibody of claim 1, wherein the antibody is monoclonal.

3. The antibody of claim 1, wherein the antibody is humnanized.

4. The antibody of claim 1, wherein said interferon gamma inducing factor is IL-18.

5. A method of inducing protective immunity against a T-cell mediated autoimmune disease, the method comprising administering to an individual having the T-cell mediated autoimmune disease or being predisposed thereto, cells being capable of producing and secreting an antibody capable of neutralizing an interferon gamma inducing factor thereby inducing protective immunity against the T-cell mediated autoimmune disease in said individual.

6. The method of claim 5, wherein the antibody is humanized.

7. The method of claim 5, wherein the T-cell mediated autoimmune disease is selected from the group consisting of multiple sclerosis, rheumatoid arthritis, type I diabetes, uveoretinitis, Crohn's disease and ulcerative colitis.

8. A method of inducing protective immunity against a T-cell mediated autoimmune disease, the method comprising administering to an individual having the T-cell mediated autoimmune disease or being predisposed thereto, an antibody capable of neutralizing an interferon gamma inducing factor thereby inducing protective immunity against the T-cell mediated autoimmune disease in said individual.

9. The method of claim 8, wherein the antibody is humanized.

10. The method of claim 8, wherein the T-cell mediated autoimmune disease is selected from the group consisting of multiple sclerosis, rheumatoid arthritis, type I diabetes, uveoretinitis, Crohn's disease and ulcerative colitis.

11. An article-of-manufacturing comprising packaging material and a pharmaceutical composition being identified for use in inducing protective immunity against a T-cell mediated autoimmune disease, said pharmaceutical composition comprising a pharmaceutically acceptable carrier and an antibody being capable of binding an interferon gamma inducing factor.

12. The article-of-manufacturing of claim 11, wherein the antibody is humanized.

13. The method of claim 11, wherein the T-cell mediated autoimmune disease is selected from the group consisting of multiple sclerosis, rheumatoid arthritis, type I diabetes, uveoretinitis, Crohn's disease and ulcerative colitis.

14. A method of inducing protective immunity against multiple sclerosis, the method comprising administering to an individual having the T-cell mediated autoimmune disease or being predisposed thereto, a nucleic acid construct including a polynucleotide sequence encoding a polypeptide being capable of eliciting in said individual formation of antibodies capable of neutralizing an interferon gamma inducing factor thereby inducing protective immunity against multiple selerosis in said individual.

15. The method of claim 14, wherein said nucleic acid construct also includes one or more transcription control sequences operatively linked to said polynucleotide sequence.

16. The method of claim 15, wherein said transcription control sequences are selected from the group consisting of RSV control sequences, CMV control sequences, retroviral LTR sequences, SV-40 control sequences and β-actin control sequences.

17. The method of claim 14, wherein said nucleic acid construct is an eukaryotic expression vector.

18. The method of claim 14, wherein said nucleic acid construct is selected from the group consisting of pcDNA3, pcDNA3.1(+/−), pZeoSV2(+/−), pSecTag2, pDisplay, pEF/myc/cyto, pCMV/myc/cyto, pCR3.1, pCI, pBK-RSV, pBK-CMV, pTRES and their derivatives.

19. The method of claim 14, wherein said nucleic acid construct is administered to said individual parenterally.

20. The method of claim 14, wherein said individual is a human being.

21. The method of claim 14, wherein said nucleic acid construct is administered to said individual via a viral vector.

22. A method of inducing protective immunity against a T-cell mediated autoimmune disease, the method comprising:
(a) obtaining cells of an individual;
(b) introducing into said cells a nucleic acid construct including a polynucleotide sequence encoding an interferon gamma inducing factor or an immunogenic portion thereof to thereby generate genetically modified cells expressing and optionally secreting said interferon gamma inducing factor or said immunogenic portion thereof; and
(c) reintroducing said genetically modified cells to said individual thereby inducing protective immunity against the T-cell mediated autoimmune disease in said individual.

23. The method of claim 22, wherein said nucleic acid construct also includes one or more transcription control sequences operatively linked to said polynucleotide sequence.

24. The method of claim 23, wherein said transcription control sequences are selected from the group consisting of RSV control sequences, CMV control sequences, retroviral LTR sequences, SV-40 control sequences and β-actin control sequences.

25. The method of claim 22, wherein said nucleic acid construct is an eukaryotic expression vector.

26. The method of claim 22, wherein said nucleic acid construct is selected from the group consisting of pcDNA3, pcDNA3.1(+/−), pZeoSV2(+/−), pSecTag2, pDisplay, pEF/myc/cyto, pCMV/myc/cyto, pCR3.1, pCI, pBK-RSV, pBK-CMV, pTRES and their derivatives.

27. The method of claim 22, wherein said individual is a human being.

28. The method of claim 22, wherein the T-cell mediated autoimmune disease is selected from the group consisting of multiple sclerosis, rheumatoid arthritis, type I diabetes, uveoretinitis, Crohn's disease and ulcerative colitis.

29. An article-of-manufacturing comprising packaging material and a pharmaceutical composition being identified for use in inducing protective immunity against a T-cell mediated autoimmune disease, said pharmaceutical composition comprising a pharmaceutically acceptable carrier and a nucleic acid construct including a polynucleotide sequence encoding a polypeptide being capable of eliciting formation of antibodies capable of neutralizing an interferon gamma inducing factor.

30. The article-of-manufacturing of claim 29, wherein said pharmaceutically acceptable carrier is selected from the group consisting of an aqueous physiologically balanced solution, an artificial lipid-containing substrate, a natural lipid-containing substrate, an oil, an ester, a glycol, a virus and metal particles.

31. The article-of-manufacturing of claim 29, wherein said pharmaceutically acceptable carrier comprises a delivery vehicle that delivers said nucleic acid construct to said individual.

32. The article-of-manufacturing of claim 31, wherein said delivery vehicle is selected from the group consisting of liposomes, micelles, and cells.

33. The article-of-manufacturing of claim 29, wherein said nucleic acid construct is an eukaryotic expression vector.

34. The article-of-manufacturing of claim 29, wherein said pharmaceutical composition is formulated suitable for parenteral administration to a human.

35. The method of claim 29, wherein the T-cell mediated autoimmune disease is selected from the group consisting of multiple sclerosis, rheumatoid arthritis, type I diabetes, uveoretinitis, Crohn's disease and ulcerative colitis.

36. The use of an anti interferon gamma inducing factor antibody in the treatment of a T-cell mediated autoimmune disease.

37. The method of claim 36, wherein the T-cell mediated autoimmune disease is selected from the group consisting of multiple sclerosis, rheumatoid arthritis, type I diabetes, uveoretinitis, Crohn's disease and ulcerative colitis.
